(19) United States
(12) Patent Application Publication
(10) Pub. No.: US 2024/0044899 A1
(43) Pub. Date: Feb. 8, 2024

(54) BIOMARKERS FOR FIMEPINOSTAT THERAPY

(71) Applicant: Curis, Inc, Lexington, MA (US)

(72) Inventors: Mariano J. ALVAREZ, El Cajon, CA (US); Yao Shen, Fort Lee, NJ (US)

(21) Appl. No.: 18/226,984

(22) Filed: Jul. 27, 2023

Related U.S. Application Data
(63) Continuation of application No. PCT/US22/14670, filed on Feb. 1, 2022.
(60) Provisional application No. 63/145,128, filed on Feb. 3, 2021.

(51) Int. Cl. G01N 33/574; A61K 31/5377; A61P 35/00

(52) U.S. Cl. CPC G01N 33/57407; A61K 31/5377; A61P 35/00

(57) ABSTRACT

The present invention provides biomarkers for identifying patients suffering from diffuse large B-cell lymphoma who are likely to respond to therapy with fimepinostat. The invention further provides methods for treating such patients with fimepinostat.

### RELATED APPLICATIONS

This application is a continuation of International Application No. PCT/US22/14670, which designated the United States and was filed on Feb. 1, 2022, published in English, which claims the benefit of U.S. Provisional Application No. 63/145,128, filed on Feb. 3, 2021. The entire teachings of the above applications are incorporated herein by reference.

### BACKGROUND OF THE INVENTION

Diffuse large B cell lymphoma (DLBCL) and high grade B cell lymphoma (HGBL) are forms of aggressive B cell non-Hodgkin lymphoma, and patients diagnosed with these diseases respond variably to first-line immunochemotherapy as well as salvage immunochemotherapy followed by high dose chemotherapy with autologous stem cell transplantation (HDC/ASCT) in the second-line setting. While clinical outcomes for patients with DLBCL have traditionally been predicted by the International Prognostic Index score in both settings as well as interval of first remission for those receiving salvage immunochemotherapy, more recently it has become apparent that immunohistochemical and molecular features of these lymphomas can serve as both prognostic and predictive biomarkers.

MYC is a human proto-oncogene which serves as a transcription factor regulating the control of cellular activities, particularly cell cycle activation.^{1,2} In DLBCL/HGBL, described MYC abnormalities include rearrangement/translocation, copy number gain/amplification and mutation. MYC translocation/rearrangement has been shown to predict for inferior survival in patients with newly-diagnosed DLBCL when treated with rituximab, cyclophosphamide, doxorubicin, vincristine and prednisone (R-CHOP)^{3,4} as well as those with R/R DLBCL following receipt of salvage immunochemotherapy with or without subsequent HDC/ASCT.⁵ While outcomes for patients diagnosed with a subgroup of MYC-rearranged/translocated HGBLs characterized by concomitant rearrangements in BCL2 and/or BCL6, known as double hit lymphomas (DHL), may be improved following receipt of intensive first-line immunochemotherapy,⁶ long-term survival is uncommon in the relapsed/refractory (R/R) setting.^{5,7} Additionally, increased copy number of MYC is also associated with a poor prognosis following receipt of first-line immunochemotherapy.^{8,9}

Independent of MYC abnormalities, increased expression of MYC protein by immunohistochemical staining (IHC) is also predictive of inferior survival in patients with newly-diagnosed DLBCL when treated with R-CHOP.⁸⁻¹⁰ The same is true for those patients whose DLBCL demonstrates increased expression of both MYC and BCL2 proteins, known as double expressor lymphoma (DEL). 11-13 Patients with DEL are also unlikely to achieve prolonged survival following salvage immunochemotherapy and HDC/ASCT.⁷ Interestingly, the poor prognosis of patients with newly-diagnosed DLBCL with activated B cell (ABC) subtype as compared to those with germinal center B (GCB) subtype, who are treated with R-CHOP, may be due to the high proportion of DEL within the ABC subtype, as survival outcomes do not differ by subtype in patients with non-DEL DLBCL in this clinical setting.¹³

Approximately ⅓ of patients with newly-diagnosed DLBCL/HBGL harbor the “MYC alterations” of MYC rearrangement/translocation and/or expression of MYC protein ≥40% by IHC.¹⁴ While the incidence of MYC alterations in patients with the R/R DLBCL/HGBL has not been clearly reported, it is reasonable to assume that it is likely greater than reported in the newly-diagnosed setting given the high probability of treatment failure as mentioned above. Therefore, due to both the high frequency of MYC alterations in patients diagnosed with DLBCL/HGBL as well as poor clinical outcomes experienced by these patients when treated with standard curative-intent regimens, additional therapeutic options for these patients are needed.

Fimepinostat (CUDC-907) is a first-in-class oral small molecule inhibitor of histone deactylase (HDAC) class I and II as well as phosphatidylinositol 3-kinase (PI3K) α, β and δ enzymes. HDAC inhibition leads to decreased transcription of MYC and translation of MYC messenger ribonucleic acid (mRNA) while PI3K inhibition leads to enhancement of ubiquitin-mediated MYC protein degradation. Treatment with fimepinostat has resulted in superior preclinical activity in DLBCL xenografts with MYC alterations, as compared to treatment with HDAC or PI3K inhibitor monotherapy.¹⁵

Fimepinostat was first studied in patients with R/R lymphoma or multiple myeloma in the phase 1 setting (NCT01742988),¹⁶ and a subgroup analysis of 11 evaluable DLBCL patients with MYC-altered disease as defined by central or local testing demonstrated a 64% overall response rate and estimated 13.6 month duration of response.¹⁷ Subsequently, a phase 2 protocol of fimepinostat for patients with DLBCL, including those with MYC alterations, was developed (NCT02674750). Here, we report the clinical outcomes and safety profile of fimepinostat in patients with MYC-altered disease as defined by central testing who were enrolled in these protocols.

There is a need for biomarkers and methods of use thereof for selecting lymphoma patients who are most likely to respond to fimepinostat therapy.

### SUMMARY OF THE INVENTION

The present invention relates to methods of determining if a subject suffering from diffuse large B cell lymphoma (“DLBCL”) who is expected to be responsive to treatment with fimepinostat (a fimepinostat responder) or non-responsive to treatment with fimepinostat (a fimepinostat non-responder). The structure of fimepinostat is shown below.

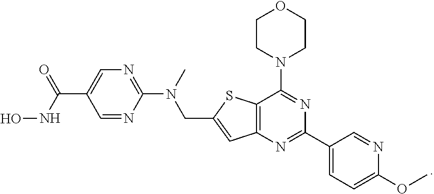 [SMILES: COc1ccc(-c2nc(N3CCOCC3)c3sc(CN(C)c4ncc(C(=O)NO)cn4)cc3n2)cn1]

In one embodiment, the invention provides a method of classifying a subject suffering from DLBCL as a fimepinostat responder or a fimepinostat non-responder, comprising determining a plurality of protein activity values in the subject, wherein each protein activity value corresponds to one of a set of proteins in the subject; providing the plurality of protein activity values to a trained classifier, the trained classifier being trained to differentiate between fimepinostat responders and fimepinostat non-responders to fimepinostat therapy; and obtaining from the classifier a classification of the subject as a fimepinostat responder or a fimepinostat non-responder.

In one embodiment, the present invention provides a method of classifying a subject suffering from DLBCL as a fimepinostat responder or a fimepinostat non-responder. The method comprises determining the activity of one or more marker proteins, such as Master Regulator proteins, in a tumor sample from the subject, wherein increased activity of the one or more marker proteins compared to baseline identifies the subject as a fimepinostat responder and an absence of increased activity of the one or more marker proteins compared to baseline identifies the subject as a fimepinostat non-responder. The baseline activity of the marker proteins can be, for example, determined as the average across a set of control samples, such as a set of control tumor samples. In certain embodiments, the control samples comprise 1000, 5000, 7500, 10000, 12000 or more tumor samples.

In one embodiment, the present invention provides a method of treating a subject suffering from DLBCL, wherein the subject is a fimepinostat responder. The method comprises the step of administering to the subject a therapeutically effective amount of fimepinostat or a pharmaceutically acceptable salt thereof.

In another embodiment, the present invention provides a method of treating DLBCL in a subject in need thereof, comprising the steps of (1) classifying the subject as a fimepinostat responder or a fimepinostat non-responder and (2) if the subject is a fimepinostat responder, administering to the subject a therapeutically effective amount of fimepinostat or a pharmaceutically acceptable salt thereof. Preferably, the method further provides the step of administering to the subject a therapeutically effective amount of a therapy for DLBCL which is not fimepinostat or a pharmaceutically acceptable salt thereof if the subject is classified as a fimepinostat non-responder.

In another embodiment, the present invention provides a method of treating DLBCL in a subject in need thereof, wherein the subject is a fimepinostat responder, comprising the steps of (1) receiving information identifying the subject as a fimepinostat responder; and (2) administering to the subject a therapeutically effective amount of fimepinostat or a pharmaceutically acceptable salt thereof.

In another example embodiment, the present invention provides a computer program product for classifying a subject suffering from DLBCL as a fimepinostat responder or non-responder. The computer program product comprises a computer readable storage medium having program instructions embodied thereon, wherein the program instructions are executable by a computer processor to perform a method comprising determining a plurality of protein activity values in the subject, each protein activity value corresponding to one of a set of proteins in the subject; providing the plurality of protein activity values to a classifier which is trained to differentiate between fimepinostat responders and non-responders; and obtaining from the classifier a classification of the subject as a fimepinostat responder or non-responder.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows the patient selection process for the Phase I and Phase II clinical trials described in the Exemplification.

FIG. 2A is a graph of progression free survival observed in the Phase I and Phase II trials described in the Exemplification.

FIG. 2B is a graph of overall survival observed in the Phase I and Phase II trials described in the Exemplification.

FIG. 2C is a graph of duration of response observed in the Phase I and Phase II trials described in the Exemplification.

FIG. 3 is a graph showing the results of Gene Set Enrichment Analysis [GSEA] of 67 MYC-interacting proteins (vertical lines) in the list of proteins sorted from the most to the least differentially active between fimepinostat responder and non-responder patients in the Phase I and Phase II trials described in the Exemplification.

FIG. 4A is a heatmap showing the Virtual Inference of Protein activity by Enriched Regulon analysis (VIPER)-inferred activity for the three fimepinostat response master regulatory (MR) proteins used by the biomarkers (rows) for all samples. The clinical samples included in the analysis (columns) were rank-sorted based on the predicted likelihood of response by the NN-biomarker (barplot above the heatmap), estimated using leave-one—out cross validation (LOO-CV). Patients that responded to fimepinostat (complete response (CR) and partial response (PR)) and patients that did not respond to fimepinostat (progressive disease (PD)) are shown in black and white, respectively (clinical benefit row). Patients with MYC-altered disease are indicated in black in the MYC-altered row.

FIG. 4B is a graph showing the results of a Receiver Operating Characteristic (ROC) analysis for the LOO-CV performed on all samples (n=22). Area under the ROC Curve (AUC) and its 95% confidence interval (CI) are shown inside the plot.

FIG. 4C is a graph showing the results of a Receiver Operating Characteristic (ROC) analysis for the LOO-CV performed on the MYC altered samples (n=13). Area under the ROC Curve (AUC) and its 95% confidence interval (CI) are shown inside the plot.

### DETAILED DESCRIPTION OF THE INVENTION

The present invention provides a biomarker which is predictive of response to fimepinostat therapy and methods of using this biomarker for classifying a subject suffering from DLBCL as a fimepinostat responder or a fimepinostat non-responder. The invention further provides methods of treating DLBCL in a subject in need thereof following classification of the subject as a fimepinostat responder.

### Determination of Protein Activity

In various embodiments, protein activity is determined for the one or more subjects based on genetic data. Protein activity for a population of subjects is used to identify MR proteins as described above, and to train classifiers based on sets of known responders and non-responders. Similarly, protein activity for an individual subject is used to classify that subject as a responder or non-responder, based on the inference of the response likelihood. In particular, a feature vector is constructed for a given subject that comprises protein activity values for one or more proteins.

Various measures of protein activity are suitable for use according to the present disclosure. For example, as described further below, VIPER provides protein activity values in terms of normalized enrichment scores, which express activity for all the regulatory proteins in the same scale. However, it will be appreciated that alternative methods of determining protein activity provide alternative measures of protein activity values, for example, absolute or relative abundance in a sample, or absolute enrichment.

Various embodiments described herein employ the VIPER algorithm to determine protein activity in the form of normalized enrichment scores for a plurality of proteins based on a predetermined model of transcriptional regulation. The VIPER algorithm is described further in WO 2017/040311 and U.S. Pat. No. 10,790,040 B2, each of which is hereby incorporated by reference in its entirety.

It will be appreciated that alternative methods of determining protein activity in a subject are also applicable for practicing the methods described herein. Exemplary alternative algorithms for inferring protein activity from gene expression data include: ChIP-X Enrichment Analysis (ChEA), which is described further in Keenan, A. B. et al. ChEA3: transcription factor enrichment analysis by orthogonal omics integration. Nucleic Acids Res. 47, W212-W224 (2019); TFEA.ChIP, which is described further in Puente-Santamaria, L., Wasserman, W. W. & Del Peso, L. TFEA.ChIP: a tool kit for transcription factor binding site enrichment analysis capitalizing on ChIP-seq datasets. Bioinformatics 35, 5339-5340 (2019); Binding Analysis for Regulation of Transcription (BART), which is described further in Wang, Z. et al. BART: a transcription factor prediction tool with query gene sets or epigenomic profiles. Bioinformatics 34, 2867-2869 (2018); Mining Gene Cohorts for Transcriptional Regulators Inferred by Kolmogorov-Smirnov Statistics (MAGICTRICKS), which is described further in Roopra A. MAGICTRICKS: A tool for predicting transcription factors and cofactors that drive gene lists. https://doi.org/10.1101/492744; DoRothEA, which is described further in Garcia-Alonso, L. et al. Transcription factor activities enhance markers of drug sensitivity in cancer. Cancer Res. 78, 769-780 (2018); and NetFactor, which is described further in Ahsen, M. E. et al. NeTFactor, a framework for identifying transcriptional regulators of gene expression-based biomarkers. Sci. Rep. 9, 12970 (2019).

In addition, biochemical approaches can be used to estimate abundance of the proteins included in a given biomarker, such us immunostaining (immunofluorescence or immunochemistry) of tissue samples followed by histological examination, flow cytometry, mass cytometry or cytometric bead arrays, reverse-phase protein arrays, bead-based IVD assays such as Luminex and mass spectrometry.

### Methods of Classifying a Subject as a Fimepinostat Responder or Non-Responder

A set of MR proteins may be determined by a variety of methods, including those described in connection with the examples below. For example, cluster analysis may be performed with or without separate dimensionality reduction in order to determine the heterogeneity of responder and non-responder clusters in an n-dimensional vector space, with n corresponding to a number of proteins considered. It will be appreciated that a variety of methods are available for dimensionality reduction, including unsupervised dimensionality reduction techniques such as principal component analysis (PCA), random projection, and feature agglomeration analysis. It will further be appreciated that a variety of cluster analysis methods are available, including hierarchical clustering and k-means clustering. It will be appreciated that a variety of statistical methods are available for determining the correlation of a given protein value to the classification as a responder or non-responder.

In various embodiments described, the DarwinOncoTarget™ system is used to identify and rank potential protein predictors of responsiveness and non-responsiveness. Table 1 provides a listing of the top 10 proteins showing differential activity between responder and non-responder patients, sorted by the False Discovery Rate (FDR)-corrected p-value. The first three of this list provide the exemplary biomarker described herein.

In various embodiments, a subset of proteins is selected by performing a cross-validation process such as leave-one-out cross validation. In such embodiments, a model is trained on all data except for one point and a prediction is made for that point. It will be appreciated that cross-validation may be used to optimize the selection of proteins and/or the number of proteins. In addition, repeated application of cross-validation may be employed with multiple models in order to select an optimal pairing of model and proteins. Accordingly, it will be appreciated that a variable number of proteins may be selected for training a classifier as set out herein. In particular, in various embodiments, any subset of the MR proteins provided in Table 1 may be used to train one or more classifiers. It will be appreciated that while there may be computational advantages to reduction in the number of MR proteins used to train a given classifier, a classifier may be trained with all or some of the potential proteins while still arriving at a trained classifier suitable for identification of responders and non-responders. In particular, while inclusion of additional low value proteins may increase training time, a given classifier will de-emphasize low value proteins while emphasizing high value proteins by virtue of the training process. In some embodiments, a predetermined number of proteins having the highest differential activity between responder and non-responder patients are selected.

A training set including responders and non-responders is determined by RNA sequencing of a plurality of subjects. Normalized enrichment scores (NES) are determined for a plurality of proteins across the training set. In some embodiments, normalized enrichment scores are determined by application of VIPER.

During a training phase according to various embodiments, protein activity scores for responsive and non-responsive subjects are determined as set forth above. A feature vector is constructed for each of the responsive and non-responsive subjects, and provided to a classifier. In some embodiments, the classifier comprises a SVM. In some embodiments, the classifier comprises an artificial neural network. In some embodiments, the classifier comprises a random decision forest. It will be appreciated that a variety of other classifiers are suitable for use according to the present disclosure, including linear classifiers, support vector machines (SVM), Linear Discriminant Analysis (LDA), Logistic regression, Random Forest, Ridge regression methods, or neural networks such as recurrent neural networks (RNN). In addition, it will be appreciated that an ensemble model including any combination of the foregoing may also be employed.

Suitable artificial neural networks include but are not limited to a feedforward neural network, a radial basis function network, a self-organizing map, learning vector quantization, a recurrent neural network, a Hopfield network, a Boltzmann machine, an echo state network, long short-term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

Based upon the training set, the classifier is trained to estimate the likelihood, as a number between 0 and 1, for a subject to be responsive, which can be used to classify such subject as either responsive or non-responsive.

In a classification phase according to various embodiments, a protein activity of a given subject is determined. The protein activity values are provided as a feature vector to a trained classifier, which provides as output the estimated likelihood for the subject to be a responder.

A biomarker predictive of response to fimepinostat was sought in DLBCL patients treated with fimepinostat using the VIPER algorithm as discussed above. VIPER analysis was performed on pretreatment tumor biopsies from 11 fimepinostat responders and 11 fimepinostat non-responders from the Phase I and Phase II clinical trials described in the Exemplification. For this analysis, subjects who achieved a partial response (PR) or a complete response (CR) to fimepinostat therapy were deemed responders, while those who exhibited progressive disease (PD) were deemed non-responders.

The top 10 proteins showing differential activity between fimepinostat responders and non-responders are set forth in Table 1 below and are sorted by the False Discovery Rate (FDR)-corrected p-value.

| EntrezID | GeneSymbol | FDR | Description |
|---|---|---|---|
| 57326 | PBXIP1 | 1.07E−18 | pre-B-cell leukemia homeobox interacting protein 1 |
| 2113 | ETS1 | 9.02E−11 | v-ets erythroblastosis virus E26 oncogene homolog 1 (avian) |
| 27329 | ANGPTL3 | 2.49E−06 | angiopoietin-like 3 |
| 8089 | YEATS4 | 1.25E−05 | YEATS domain containing 4 |
| 3603 | IL16 | 3.66E−05 | interleukin 16 (lymphocyte chemoattractant factor) |
| 89846 | FGD3 | 3.66E−05 | FYVE, RhoGEF and PH domain containing 3 |
| 55729 | ATF7IP | 7.67E−05 | activating transcription factor 7 interacting protein |
| 9319 | TRIP13 | 7.80E−05 | thyroid hormone receptor interactor 13 |
| 8535 | CBX4 | 7.80E−05 | chromobox homolog 4 |
| 951 | CD37 | 7.80E−05 | CD37 molecule |

In certain embodiments, a subject is classified or identified as a fimepinostat responder or a fimepinostat non-responder by determining the protein activity of any one of the proteins in Table 1 or a combination of two or more thereof. Preferably, a subject is classified or identified as a fimepinostat responder or a fimepinostat non-responder by determining the protein activity of a combination of three or more proteins in Table 1.

In preferred embodiments, the one or more proteins whose activities are used to classify a subject as a fimepinostat responder or a fimepinostat non-responder in the methods of the invention are one, two or three of the following MR proteins:

Human PBXIP1, Pre-B-cell leukemia transcription factor-interacting protein 1, described, for example, as UniProtKB: Q96AQ6 at the URL https://www.uniprot.org/uniprot/Q96AQ6.
Human ETS1, Protein C-ets-1, described, for example, as UniProtKB P14921 at the URL https://www.uniprot.org/uniprot/P14921.
Human ANGPTL3, Angiopoietin-related protein 3, described, for example, as UniProtKB Q9Y5C1 at the URL https://www.uniprot.org/uniprot/Q9Y5C1.

In preferred embodiments, a subject is classified or identified as a fimepinostat responder or a fimepinostat non-responder by determining the activity of all three of the proteins PBXIP1, ETS1 and AGPTL3, which were determined to be differentially active in responders compared to non-responders and were identified as Master Regulators of sensitivity to fimepinostat. In the study described herein, these three proteins produced optimal predictive performance based on leave-1-out cross-validation (area under receiver operating characteristic curve). The 3-protein classifier correctly identified 9 of 11 responders (82%) and incorrectly classified 2 of 11 non-responders (18%).

The classification of a subject can be implemented using a suitable computer program for identifying fimepinostat responders and fimepinostat non-responders. The computer program is preferably embodied on a computer readable storage medium and comprises program instructions which are executable by a processor to cause the processor to perform a method comprising determining a plurality of protein activity values in a subject suffering from DLBCL, each protein activity value corresponding to one or more of the proteins PBXIP1, ETS1 and AGPTL3; providing the plurality of protein activity values to a trained classifier, the trained classifier being trained to differentiate between fimepinostat responders and fimepinostat non-responders; and obtaining from the classifier a classification of the subject as a fimepinostat responder or a fimepinostat non-responder.

### Methods of Treating DLBCL

In one embodiment, the present invention provides a method of treating a patient suffering from DLBCL, comprising determining protein activity values of one or more of PBXIP1, ETS1 and AGPTL3 in biopsied tumor tissue from the subject; classifying the subject as a responder or non-responder to fimepinostat treatment; and, if the subject is classified as a fimepinostat responder, administering to the subject a therapeutically effective amount of fimepinostat or a pharmaceutically acceptable salt thereof.

In another embodiment, the present invention provides a method of treating a subject suffering from DLBCL, comprising administering to the subject a therapeutically effective amount of fimepinostat or a pharmaceutically acceptable salt thereof, wherein the subject is a fimepinostat responder.

In another embodiment, the present invention is a method of treating a subject suffering from DLBCL, wherein the subject is classified as a fimepinostat responder, comprising administering to the subject a therapeutically effective amount of fimepinostat or a pharmaceutically acceptable salt thereof.

In another embodiment, the present invention provides a method of treating a subject suffering from DLBCL, comprising receiving information on protein activity values of one or more of PBXIP1, ETS1 and AGPTL3; and administering to the subject a therapeutically effective amount of fimepinostat or a pharmaceutically acceptable salt thereof to the subject. Preferably, the subject is only treated with fimepinonstat or a pharmaceutically acceptable salt thereof only if the subject is determined to be a fimepinostat responder.

In certain embodiments of the methods of the invention, the subject suffers from relapsed/refractory (R/R) DLBCL. In certain embodiments, the subject suffers from R/R DLBCL and has gone through at least one or two prior therapies prior to treatment with fimepinostat. In certain embodiments, the subject suffers from R/R DLBCL and has gone through 1, 2, 3 or 4 prior therapies prior to treatment with fimepinostat.

In certain embodiments, the DLBCL is of the ABC subtype or the GCB subtype. In certain embodiments, the cancer is relapsed or refractory DLBCL.

In certain embodiments of the methods of the invention, the DLBCL is a MYC-altered DLBCL. In certain embodiments, the DLBCL is a double hit or double expresser DLBCL (Quintanilla-Martinez, L., Hematol. Oncol. 2015, 33:50-55).

In the methods of treatment of the invention, fimepinostat is administered as the free base or in the form of a pharmaceutically acceptable salt. Preferably, fimepinostat is administered in the form of a pharmaceutically acceptable salt.

### Combination Therapy

In certain embodiments of the methods of treatment of the invention, the fimepinostat or pharmaceutically acceptable salt thereof may be administered in combination with one or more separate agents that modulate protein kinases involved in various disease states. Examples of such kinases may include, but are not limited to: serine/threonine specific kinases, receptor tyrosine specific kinases and non-receptor tyrosine specific kinases. Serine/threonine kinases include mitogen activated protein kinases (MAPK), meiosis specific kinase (MEK), RAF and aurora kinase. Examples of receptor kinase families include epidermal growth factor receptor (EGFR) (e.g., HER2/neu, HER3, HER4, ErbB, ErbB2, ErbB3, ErbB4, Xmrk, DER, Let23); fibroblast growth factor (FGF) receptor (e.g., FGF-R1,GFF-R2/BEK/CEK3, FGF-R3/CEK2, FGF-R4/TKF, KGF-R); hepatocyte growth/scatter factor receptor (HGFR) (e.g., MET, RON, SEA, SEX); insulin receptor (e.g., IGFI-R); Eph (e.g., CEK5, CEK8, EBK, ECK, EEK, EHK-1, EHK-2, ELK, EPH, ERK, HEK, MDK2, MDK5, SEK); Ax1 (e.g., Mer/Nyk, Rse); RET; and platelet-derived growth factor receptor (PDGFR) (e.g., PDGFa-R, PDG(3-R, CSF1-R/FMS, SCF-R/C-KIT, VEGF-R/FLT, NEK/FLK1, FLT3/FLK2/STK-1). Non-receptor tyrosine kinase families include, but are not limited to, BCR-ABL (e.g., p43^{ab1}, ARG); BTK (e.g., ITK/EMT, TEC); CSK, FAK, FPS, JAK, SRC, BMX, FER, CDK and SYK.

In another aspect of the invention, the fimepinostat or pharmaceutically acceptable salt thereof may be administered in combination with one or more separate agents that modulate non-kinase biological targets or processes. Such targets include histone deacetylases (HDAC), DNA methyltransferase (DNMT), heat shock proteins (e.g., HSP90), hedgehog pathway-related proteins (e.g., sonic hedgehog, patched, smoothened) and proteosomes.

In certain embodiments, the fimepinostat or pharmaceutically acceptable salt thereof may be combined with a BCL2 inhibitor, such as venetoclax. In this embodiment, the DLBCL is preferably a MYC-altered DLBCL, a double hit DLBCL or a double expresser DLBCL.

In certain embodiments, the fimepinostat or pharmaceutically acceptable salt thereof may be combined with antineoplastic agents (e.g., small molecules, monoclonal antibodies, antisense RNA, and fusion proteins) that inhibit one or more biological targets such as Zolinza, Tarceva, Iressa, Tykerb, Gleevec, Sutent, Sprycel, Nexavar, Sorafinib, CNF2024, RG108, BMS387032, Affinitak, Avastin, Herceptin, Erbitux, AG24322, PD325901, ZD6474, PD184322, Obatodax, ABT737, GDC-0449, IPI-926, BMS833923, LDE225, PF-04449913 and AEE788. Such combinations may enhance therapeutic efficacy over efficacy achieved by any of the agents alone and may prevent or delay the appearance of resistant mutational variants.

In certain preferred embodiments, the fimepinostat or pharmaceutically acceptable salt thereof is administered in combination with a chemotherapeutic agent. Chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as mustard gas derivatives (Mechlorethamine, cyclophosphamide, chlorambucil, melphalan, ifosfamide), ethylenimines (thiotepa, hexamethylmelanine), Alkylsulfonates (Busulfan), Hydrazines and Triazines (Altretamine, Procarbazine, Dacarbazine and Temozolomide), Nitrosoureas (Carmustine, Lomustine and Streptozocin), Ifosfamide and metal salts (Carboplatin, Cisplatin, and Oxaliplatin); plant alkaloids such as Podophyllotoxins (Etoposide and Tenisopide), Taxanes (Paclitaxel and Docetaxel), Vinca alkaloids (Vincristine, Vinblastine, Vindesine and Vinorelbine), and Camptothecan analogs (Irinotecan and Topotecan); anti-tumor antibiotics such as Chromomycins (Dactinomycin and Plicamycin), Anthracyclines (Doxorubicin, Daunorubicin, Epirubicin, Mitoxantrone, Valrubicin and Idarubicin), and miscellaneous antibiotics such as Mitomycin, Actinomycin and Bleomycin; anti-metabolites such as folic acid antagonists (Methotrexate, Pemetrexed, Raltitrexed, Aminopterin), pyrimidine antagonists (5-Fluorouracil, Floxuridine, Cytarabine, Capecitabine, and Gemcitabine), purine antagonists (6-Mercaptopurine and 6-Thioguanine) and adenosine deaminase inhibitors (Cladribine, Fludarabine, Mercaptopurine, Clofarabine, Thioguanine, Nelarabine and Pentostatin); topoisomerase inhibitors such as topoisomerase I inhibitors (Ironotecan, topotecan) and topoisomerase II inhibitors (Amsacrine, etoposide, etoposide phosphate, teniposide); monoclonal antibodies (Alemtuzumab, Gemtuzumab ozogamicin, Rituximab, Trastuzumab, Ibritumomab Tioxetan, Cetuximab, Panitumumab, Tositumomab, Bevacizumab); and miscellaneous anti-neoplastics such as ribonucleotide reductase inhibitors (Hydroxyurea); adrenocortical steroid inhibitor (Mitotane); enzymes (Asparaginase and Pegaspargase); anti-microtubule agents (Estramustine); retinoids (Bexarotene, Isotretinoin, Tretinoin (ATRA), and Lenalidomide.

In certain preferred embodiments, the fimepinostat or pharmaceutically acceptable salt thereof is administered in combination with a chemoprotective agent. Chemoprotective agents act to protect the body or minimize the side effects of chemotherapy. Examples of such agents include, but are not limited to, amfostine, mesna, and dexrazoxane.

In one aspect of the invention, the fimepinostat or pharmaceutically acceptable salt thereof is administered in combination with radiation therapy. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

It will be appreciated that the fimepinostat or pharmaceutically acceptable salt thereof can be used in combination with an immunotherapeutic agent. One form of immunotherapy is the generation of an active systemic tumor-specific immune response of host origin by administering a vaccine composition at a site distant from the tumor. Various types of vaccines have been proposed, including isolated tumor-antigen vaccines and anti-idiotype vaccines. Another approach is to use tumor cells from the subject to be treated, or a derivative of such cells (reviewed by Schirrmacher et al., (1995) J. Cancer Res. Clin. Oncol. 12 1:487). In U.S. Pat. No. 5,484,596, Hanna Jr., et al. claim a method for treating a resectable carcinoma to prevent recurrence or metastases, comprising surgically removing the tumor, dispersing the cells with collagenase, irradiating the cells, and vaccinating the patient with at least three consecutive doses of about 10′ cells.

### Pharmaceutical Compositions

In preferred embodiments, fimepinostat or a pharmaceutically acceptable salt thereof is administered to the subject in a pharmaceutical composition. The pharmaceutical composition comprises a therapeutically effective amount of fimepinostat or a pharmaceutically acceptable salt thereof formulated together with one or more pharmaceutically acceptable carriers or excipients.

Preferably, the pharmaceutically acceptable carrier or excipient is a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; cyclodextrins such as alpha-(α), beta-(β) and gamma-(γ) cyclodextrins; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical composition may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the fimepinostat or pharmaceutically acceptable salt thereof, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the fimepinostat or pharmaceutically acceptable salt thereof with suitable non-irritating excipients or carriers such as cocoa butter, polyethylene glycol or a suppository wax which are solid at ambient temperature but liquid at body temperature and therefore melt in the rectum or vaginal cavity and release the active compound.

Preferred pharmaceutical compositions include solid dosage forms for oral administration such as capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragées, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of the fimepinostat or pharmaceutically acceptable salt thereof include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to fimepinostat or pharmaceutically acceptable salt thereof, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to fimepinostat or pharmaceutically acceptable salt thereof, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration (e.g., inhalation into the respiratory system). Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al., and WO 98/43650 by Montgomery, all of which are incorporated herein by reference).

In certain embodiments of the methods of the invention, the fimepinostat or pharmaceutically acceptable salt thereof is administered orally. Pharmaceutical compositions suitable for oral administration include solid forms such as pills, tablets, caplets, capsules (each including immediate release, timed release, and sustained release formulations), granules and powders; and, liquid forms such as solutions, syrups, elixirs, emulsions, and suspensions. In certain embodiments, the pharmaceutical composition is a tablet or a capsule comprising about mg (free base equivalent) of fimepinostat. In certain embodiments, the fimepinostat is present in the tablet or capsule in the form of the benzenesulfonate salt or the methanesulfonate salt.

### Definitions

The term “subject”, as used herein, is a human (i.e., a male or female of any age group, e.g., a pediatric subject (e.g., infant, child, adolescent) or adult subject (e.g., young adult, middle-aged adult or senior adult)). Preferably, a subject is an adult human.

The term “treating”, as used herein, means to decrease, suppress, attenuate, diminish, arrest, or stabilize the development or progression of a disease (e.g., a disease or disorder delineated herein), lessen the severity of the disease or improve the symptoms associated with the disease.

A “therapeutically effective amount of fimepinostat”, as used herein refers to an amount that is sufficient to achieve a desired therapeutic effect. For example, a therapeutically effective amount can be an amount that is sufficient to improve at least one sign or symptom of diseases or conditions disclosed herein. In a particular embodiment, the therapeutically effective amount of fimepinostat is from about 10 mg to about 200 mg. In a further particular embodiment, the therapeutically effective amount of fimepinostat is 60 mg per day. In a particular dosing regimen, fimepinostat is administered to the subject at a dose of 60 mg per day on Days 1 to 5 of each week of treatment, and no fimepinostat is administered on Days 6 and 7. The fimepinostat is preferably administered in a single daily dose of 60 mg. The fimepinostat is preferably administered orally. Fimepinostat has both acid and base functional groups, and can therefore form salts with both pharmaceutically acceptable acids or with pharmaceutically acceptable cations. When the fimepinostat is administered in the form of a pharmaceutically acceptable salt, the amounts of fimepinostat disclosed herein refer to the equivalent amount of non-ionized (free base/acid) fimepinostat.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid or inorganic acid. Examples of pharmaceutically acceptable nontoxic acid addition salts include, but are not limited to, salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid, lactobionic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl sulfonate having from 1 to 6 carbon atoms, sulfonate and aryl sulfonate. Preferred pharmaceutically acceptable salts of fimepinostat include the sodium salt, the potassium salt, the sulfate salt, the methanesulfonate salt and the benzenesulfonate salt. A particularly preferred salt of fimepinostat is the methanesulfonate salt.

The terms “Master Regulator protein(s)”, “Master Regulator(s)” and “MR protein(s)”, as used herein, are interchangeable and refer to aberrantly activated/inactivated proteins in a tissue, based on a predetermined statistical threshold, for example, at a p-value of about 0.01 or less, corrected for multiple hypothesis testing.

As used herein, the term “prior therapy” refers to a known therapy for DLBCL involving administration of one or more therapeutic agents, but does not include fimepinostat therapy. Typical prior therapies for a DLBCL patient include immunochemotherapies and a regimen consisting of rituximab, cyclophosphamide, doxorubicin, vincristine and prednisone (R-CHOP).

As used herein, the term “MYC-altered DLBCL” is a DLBCL which demonstrates increased MYC protein expression and/or MYC gene rearrangement and or MYC copy number increase.

### Exemplification

### Clinical Studies of Fimepinostat

### Study Design and Participants

Included subjects were enrolled in either the dose escalation and expansion portion of the phase 1 study evaluating fimepinsotat with or without rituximab (part 1) or the phase 2 study with fimepinostat monotherapy.

For the phase 1/part 1 study, key inclusion criteria were ≥18 years of age with histopathologically confirmed diagnosis of DLBCL or transformed follicular lymphoma (tFL) refractory to or relapsed after at least 2 prior regimens, Eastern Cooperative Oncology Group (ECOG) performance status 0-2, measurable disease on baseline radiographic assessment and adequate hematologic and organ function. Key exclusion criteria were receipt of systemic anti-cancer therapy within 3 weeks of study entry, ongoing treatment with chronic immunosuppressive therapy, active CNS lymphoma and gastrointestinal conditions which may interfere with absorption of fimepinostat. For the phase 2 study, key inclusion criteria were ≥18 years of age with histopathologically confirmed diagnosis of DLBCL, HGBL or tFL refractory to or relapsed after 2-4 prior lines of therapy for the treatment of de novo DLBCL and ineligible for (or failed) autologous or allogeneic stem cell transplant (SCT), Eastern Cooperative Oncology Group (ECOG) performance status 0-1, measurable disease on baseline radiographic assessment, confirmed availability of viable tissue (defined as most recent available archival tumor tissue, or fresh tumor samples) for central laboratory FISH and IHC testing prior to study dosing and adequate hematologic and organ function. Key exclusion criteria were receipt of systemic anti-cancer therapy within 2 weeks of study entry or experimental therapy within 5 terminal half-lives (t_{1/2}) or 4 weeks prior to enrollment, known primary mediastinal, ocular, epidural, testicular or breast lymphoma and current or planned glucocorticoid therapy with the exception of doses ≤10 mg/day prednisolone or equivalent.

Both protocols were approved by the institutional review boards of all participating centers and conducted in accordance with ethical principles that have their origin in the Declaration of Helsinki and are consistent with International Conference on Harmonization Good Clinical Practice guidelines, applicable regulatory requirements, and Curis policies.

### Procedures

Patients received fimepinostat capsules (Pharmatek Laboratories Inc., San Diego, CA, USA) orally, within 30 minutes of a meal, in 21-day cycles until disease progression was documented or other discontinuation criteria were met. In the phase 2 protocol, change in dose and/or schedule intensity of fimepinostat were allowed as per protocol due to toxicity. Safety and tolerability were assessed by the incidence and severity of adverse events as determined by NCI Common Terminology Criteria for Adverse Events (CTCAE v4.03) in both protocols.

The intention to-treat-population included al patients from both protocols who received at least one dose of fimepinostat. The evaluable population includes all patients who received at least one dose of study drug (phase 1) or one full cycle of treatment (phase 2) and completed at least one post-baseline disease assessment. Patients were re-staged according to the Revised Response Criteria for Malignant Lymphoma.¹⁸

MYC-altered disease was defined as one or more of the following results from central testing of tumor samples: expression of MYC protein in ≥40% of lymphoma cells by immunohistochemical staining (IHC), MYC rearrangement by fluorescence in situ hybridization (FISH) or >2 copies of MYC in by FISH. IHC staining of MYC (rabbit clone Y69) and BCL2 (mouse clone 124) for patients enrolled in the phase 1 study (note MYC FISH was not centrally performed in this study) were performed by Mosaic Laboratories, LLC (Lake Forest, CA, USA). IHC staining of MYC (rabbit clone Y69) and BCL2 (mouse clone 124) as well as FISH with MYC (8q24) and BCL6 (3q27) break apart probes and BCL2 (t(14;18)) fusion probe for patients enrolled in the phase 2 study were performed by NeoGenomics Laboratories, Inc (Fort Myers, FL, USA), with positive cutoff values for MYC rearrangement (>10%), MYC copy number gain (>20%), BCL2 rearrangement (≥0.5%) and BCL6 rearrangement (>10%) as defined per laboratory standard.

Outcomes of interest included overall response rate, complete response rate, median progression free survival (PFS), median overall survival (OS), median duration of response (DOR) and median time to response (TTR).

Survival times were estimated via the Kaplan Meier method and 95% confidence intervals (CI) calculated via the binomial exact method. All statistical analyses were performed using Stata version 13 (StataCorp, College Station, TX, USA).

RNASeq profiles from pre-treatment biopsies of 22 patients enrolled in the phase 1 and 2 trials were generated by Illumina sequencing. Protein activity was measured by Virtual Inference of Protein-activity by Enriched Regulon analysis (VIPER), which converts tumor sample gene-expression profiles into accurate protein activity profiles for approximately 6,000 regulatory proteins, based on the expression of their transcriptional targets (DarwinHealth).¹⁹ Unlike raw gene expression, VIPER-inferred protein activity is extremely reproducible, and this methodology (DarwinOncoTarget algorithm) has been approved by the NYS Department of Health CLIA/CLEP Validation Unit as an offering in the category of “Molecular and Cellular Tumor Markers for Oncology”²⁰ and shown to be effective for biomarker discovery.²¹ The activity of 6,213 regulatory proteins annotated as Transcription Factors (GO:0003700, or GO:0004677 and GO:0030528 or GO:0045449) or co-Transcription Factors (GO:0003712 or GO:0030528 or GO:0045449) or signaling proteins (GO:0007165 and GO:0005622 or GO:0005886) in the Gene Ontology²² was inferred by metaVIPER,²³ using transcriptional regulatory networks (interactomes) inferred by analysis of a DLBCL and an acute myeloid leukemia (AML) cohorts using the ARACNe algorithm.²⁴ MetaVIPER is an extension of the VIPER algorithm supporting integration of multiple regulatory networks. A fimepinostat-sensitivity classifier was generated by training a Neural Network²⁵ using the top k=1, . . . , 10 most differentially active proteins between responders and non-responders samples.

### Results

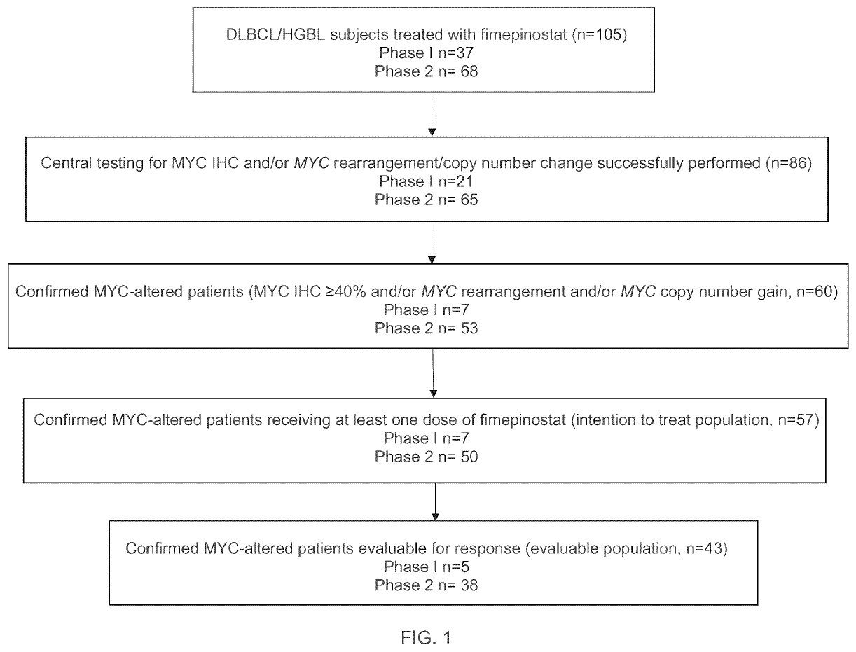

Patient identification/selection is depicted in FIG. 1. Of 105 patients with DLBCL/HGBL treated on the phase 1 and 2 protocols, 86 underwent testing for MYC protein expression and/or MYC rearrangement and/or MYC copy number gain, and 60 demonstrated one or more positive finding and were classified as exhibiting MYC-altered disease. Three patients were subsequently excluded, 2 having never been dosed and 1 receiving only 1 line of prior therapy, which resulted in an intention-to-treat population of 57 patients, all of whom received at least one dose of fimepinostat. The evaluable patient population, as defined by the phase 1 and phase 2 protocols, included 43 patients.

All patients received fimepinostat 60 mg by mouth 5 days on/2 days off as the starting dose with the exception of 1 patient enrolled in the phase 1 protocol who received 60 mg by mouth three times weekly. Three patients enrolled in the phase 1 protocol received concurrent rituximab.

Baseline characteristics of the intention-to-treat population (n=57) are described in Table 2.

TABLE 2

| Characteristic | n (%) | |
|---|---|---|
| Age (median) | 63 | years |
| Age >60 | | |
| No | 22 | (39) |
| Yes | 35 | (61) |
| Sex | | |
| Female | 22 | (39) |
| Male | 35 | (61) |
| ECOG score | | |
| 0-1 | 52 | (91) |
| 2 | 5 | (9) |
| Stage | | |
| I-II | 9 | (16) |
| III-IV | 48 | (84) |
| LDH > upper limit of normal | | |
| No | 19 | (33) |
| Yes | 37 | (66) |
| Unknown | 1 | (1) |
| Largest tumor diameter (median) | 4.7 | cm |
| Largest tumor >5 cm | | |
| No | 34 | (60) |
| Yes | 23 | (40) |
| Prior lines of therapy | | |
| 2 | 32 | (56) |
| 3 | 15 | (26) |
| ≥4 | 10 | (18) |
| Best response to last prior | | |
| therapy | | |
| Progressive disease | 28 | (49) |
| Stable disease | 7 | (12) |
| Partial response | 4 | (7) |
| Complete response | 7 | (12) |
| Unknown | 11 | (20) |
| Cell of origin by Hans | | |
| algorithm³⁰ | | |
| Non-GCB | 9 | (16) |
| GCB | 24 | (42) |
| Unknown | 24 | (42) |
| MYC IHC ≥40% | | |
| No | 4 | (7) |
| Yes | 53 | (93) |
| MYC rearrangement | | |
| No | 33 | (50) |
| Yes | 13 | (31) |
| Unknown | 11 | (19) |
| MYC increased copy number | | |
| No | 33 | (50) |
| Yes | 13 | (31) |
| Unknown | 11 | (19) |
| Double hit lymphoma (DHL) | | |
| No | 34 | (60) |
| Yes | 8 | (14) |
| Unknown | 15 | (26) |
| Double expressor | | |
| lymphoma (DEL)* | | |
| No | 5 | (10) |
| Yes | 28 | (57) |
| Unknown | 16 | (33) |

*Excluding patients with DHL
ECOG = Eastern Cooperative oncology group.
LDH = lactate dehydrogenase.
GCB = germinal center B.
IHC = immunohistochemical staining

Key characteristics include a median age of 63 years, 84% of patients with stage III-IV disease, 66% of patients with elevated lactate dehydrogenase (LDH), 40% of patients with a largest tumor diameter of ≥5 centimeters, 44% of patients having received ≥3 lines of prior therapy and 49% of patients with progressive disease as their best documented response to prior therapy. As defined by central review, 93% of tumors expressed MYC protein ≥40%, 31% demonstrated MYC rearrangement and 31% MYC copy number gain. Additionally, 14% of tumors were able to be classified as double hit lymphoma (MYC rearrangement and BCL2 and/or BCL6 rearrangement) and 57% as double expressor lymphoma (non-double hit lymphoma with MYC protein expression ≥40% and BCL2 protein expression ≥50% by IHC, respectively).^{7, 12}

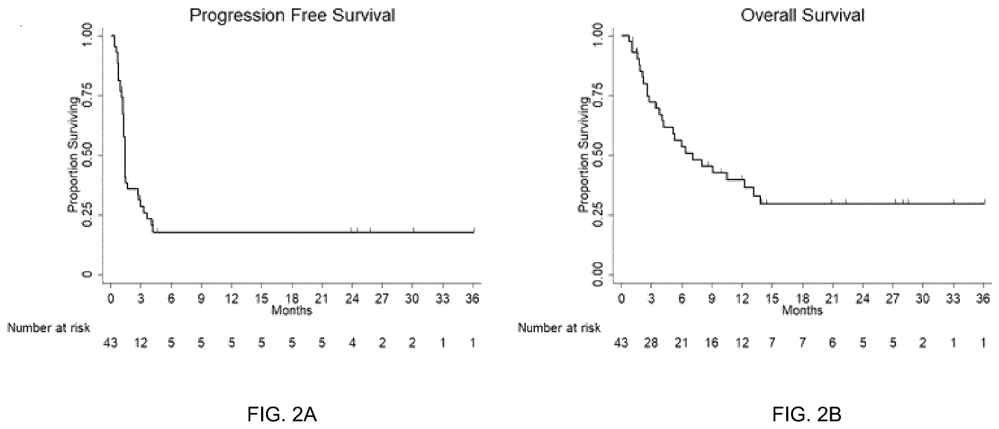

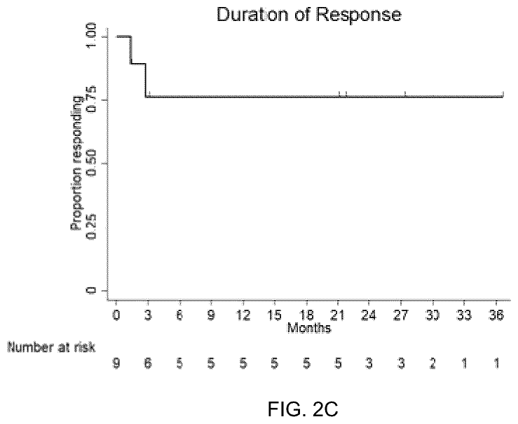

As described in Table 3, for the evaluable patient population, 9/43 patients achieved a response (21%, 95% CI 10-36%) and 6/43 patients achieved a complete response (14%, 95% CI Eight patients achieved stable disease. As depicted in FIGS. 2A, 2B and 2C, the median PFS was 1.4 months (95% CI 1.3-1.7 months), median OS 7.1 months (95% CI 3.8-13.2 months) and median DOR not yet reached (95% CI 1.4 months-not yet reached). The estimated 6 month PFS, OS and DOR were 18% (95% CI 8-31%), 54% (95% CI 37-68%) and 73% (95% CI 28-93%), respectively.

TABLE 3
Outcomes of evaluable population (n = 43)

| Outcome | Measure |
|---|---|
| Response | |
| Complete response | 6 (14%, 95% CI 5-28%) |
| Partial response | 3 (7%, 95% CI 1-19%) |
| Stable disease | 8 (19%, 95% CI 8-33%) |
| Progressive disease | 26 (60%, 95% CI 44-75%)) |
| Overall response | 9 (21%, 95% CI 10-36%) |
| Median progression free survival | 1.4 months (95% CI 1.3-1.7 months) |
| Progression free at 6 months | 18% (95% CI 8-31%) |
| Median overall survival | 7.1 months (95% CI 3.8-13.2 months) |
| Alive at 6 months | 54% (95% CI 37-68%) |
| Median duration of response | Not yet reached (95% CI 1.4 months- |
| | not yet reached) |
| Continued response at 6 months | 73% (95% CI 28-93%) |
| Median time to response | 2.8 months (95% CI 1.0-2.8 months) |

CI = confidence interval.

The median TTR was 2.8 months (95% CI 1.0-2.8 months), and 27/34 (79%) patients who ultimately experienced disease progression did so prior to 2.8 months on treatment. Of note, 1 patient achieving stable disease as best response to treatment remained on treatment for months and ultimately discontinued in favor of active observation.

Baseline characteristics and outcomes of the 9 patients responding to treatment are described in Table 4. Of note, three patients discontinued treatment while responding to proceed with cellular therapies (chimeric antigen receptor-modified T cells in 2 patients and ASCT in 1 patient) and 2 patients ultimately experienced disease progression.

TABLE 4
Selected characteristics of patients achieving partial or complete response

| | | | | | Largest | Prior | Best | % | | | | | | |
|---|---|---|---|---|---|---|---|---|---|---|---|---|---|---|
| | | | | | tumor | lines | response | MYC | | | | | | Status |
| | | | | | diam- | of | to last | expres- | MYC | | Best | TTR/ | Reason for | at |
| Subject | Age/ | ECOG | | LDH > | eter | ther- | prior | sion | RA/CN | DHL/ | re- | DOR | discon- | end of |
| ID | Sex | score | Stage | ULN | (cm) | apy | therapy | by IHC | increase | DEL | sponse | (months) | tinuation | study |
| 1 | 62/F | 1 | III | Yes | 2.0 | 2 | CR | 65 | No/ | No/ | CR | 1.2/4.1 | Inves- | Alive |
| | | | | | | | | | No | Unk | | | tigator | |
| | | | | | | | | | | | | | decision | |
| 2 | 69/M | 1 | IV | Yes | 4.9 | 3 | PR | 40 | No/ | No/ | CR | 2.7/30.0 | End of study | Alive |
| | | | | | | | | | No | Yes | | | → | |
| | | | | | | | | | | | | | compas- | |
| | | | | | | | | | | | | | sionate use | |
| 3 | 53/M | 0 | IV | Yes | 5.2 | 2 | PD | 70 | Unk/ | Unk/ | PR | 2.8/24.4 | End of study | Alive |
| | | | | | | | | | Unk | Unk | | | → CART | |
| | | | | | | | | | | | | | therapy | |
| 4 | 54/F | 0 | II | Yes | 1.9 | 2 | PD | 90 | Yes/ | Yes/ | CR | 2.8/23.8 | End of study | Alive |
| | | | | | | | | | No | NA | | | → CART | |
| | | | | | | | | | | | | | therapy | |
| 5 | 76/F | 1 | IV | No | 4.0 | 2 | PD | 80 | Unk/ | No/ | PR | 1.0/1.0 | Enroll in | Deceased |
| | | | | | | | | | Unk | Yes | | | hospice | |
| 6 | 53/F | 1 | II | No | 2.2 | 4 | Unk | 95 | No/ | No/ | PR | 2.8/4.2 | PD | Alive |
| | | | | | | | | | No | Yes | | | | |
| 7 | 52/F | 0 | IV | Yes | 3.3 | 2 | PD | 95 | Yes/ | Unk/ | CR | 1.3/4.1 | PD | Deceased |
| | | | | | | | | | Yes | Unk | | | | |
| 8 | 55/M | 0 | III | No | 1.4 | 2 | SD | 60 | Unk/ | Unk/ | CR | 1.3/2.8 | Proceed to | Alive |
| | | | | | | | | | Unk | Yes | | | SCT | |
| 9 | 46/M | 0 | III | No | 4.6 | 2 | SD | 45 | Unk/ | Unk/ | CR | 4.0/40.5 | End of study | Alive |
| | | | | | | | | | Unk | Unk | | | → | |
| | | | | | | | | | | | | | compas- | |
| | | | | | | | | | | | | | sionate use | |

ECOG = Eastern Cooperative oncology group.
LDH = lactate dehydrogenase.
IHC = immunohistochemical staining.
DHL = double hit lymphoma.
DEL = double expressor lymphoma.
TTR = time to response.
DOR = duration of response.
M = male.
F = female.
Unk = unknown.
CART = chimeric antigen receptor-modified T cell.
SCT = stem cell transplantation

Treatment-emergent adverse events (TEAE) occurring per patient by highest grade experienced with a frequency of ≥10% are listed in Table 5. The most common TEAE of any grade were diarrhea (72%), nausea (52%) and thrombocytopenia (38%). The most common grade 3 or 4 adverse events were (24%), neutropenia (15%), diarrhea (12%) and anemia (12%). Three patients experienced grade 5 TEAE: respiratory failure deemed unlikely related to treatment in 1 patient, sepsis deemed not related to treatment in 1 patient and tracheal obstruction deemed not related to treatment in 1 patient. Two non-evaluable patients discontinued treatment due to TEAE: grade 2 vomiting deemed related to treatment occurring in 1 patient and grade 4 hypercalcemia deemed unlikely related to treatment in 1 patient.

TABLE 5
Treatment-emergent adverse events (≥10% Patients)
for intention to treat population (n = 58)

| | Grades | Grade | Grade | |
|---|---|---|---|---|
| Event | 1-2 | 3 | 4 | Total |
| Diarrhea | 35 (60) | 7 (12) | 0 (0) | 42 (72) |
| Nausea | 30 (52) | 0 (0) | 0 (0) | 30 (52) |
| Thrombocytopenia | 8 (14) | 11 (19) | 3 (5) | 22 (38) |
| Fatigue | 18 (31) | 1 (2) | 0 (0) | 19 (33) |
| Decreased appetite | 18 (31) | 0 (0) | 0 (0) | 18 (31) |
| Vomiting | 15 (26) | 1 (2) | 0 (0) | 16 (28) |
| Constipation | 14 (24) | 0 (0) | 0 (0) | 14 (24) |
| Anemia | 6 (10) | 7 (12) | 0 (0) | 13 (22) |
| Hypokalemia | 7 (12) | 6 (10) | 0 (0) | 13 (22) |
| Neutropenia | 2 (3) | 7 (12) | 2 (3) | 11 (19) |
| Abdominal pain | 6 (10) | 3 (5) | 0 (0) | 9 (16) |
| Pyrexia | 9 (16) | 1 (2) | 0 (0) | 10 (17) |
| Cough | 9 (16) | 0 (0) | 0 (0) | 9 (16) |
| Dyspnea | 6 (10) | 3 (5) | 0 (0) | 9 (16) |
| Dizziness | 8 (14) | 0 (0) | 0 (0) | 8 (14) |
| Weight decreased | 7 (12) | 1 (2) | 0 (0) | 8 (14) |
| Arthralgia | 7 (12) | 0 (0) | 0 (0) | 7 (12) |
| Hypomagnesaemia | 7 (12) | 0 (0) | 0 (0) | 7 (12) |
| Pain in extremity | 6 (10) | 1 (2) | 0 (0) | 7 (12) |
| White blood cell count decreased | 3 (5) | 3 (5) | 1 (2) | 7 (12) |
| Back pain | 5 (9) | 1 (2) | 0 (0) | 6 (10) |
| Hypophosphatemia | 2 (3) | 4 (7) | 0 (0) | 6 (10) |
| Lymphocyte count decreased | 3 (5) | 3 (5) | 0 (0) | 6 (10) |
| Edema peripheral | 6 (10) | 0 (0) | 0 (0) | 6 (10) |
| Upper respiratory tract infection | 6 (10) | 0 (0) | 0 (0) | 6 (10) |

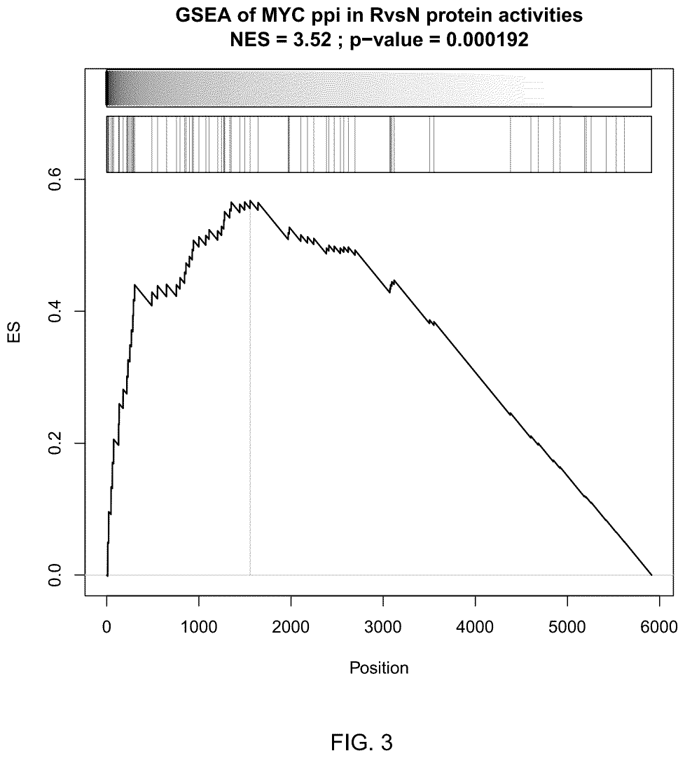

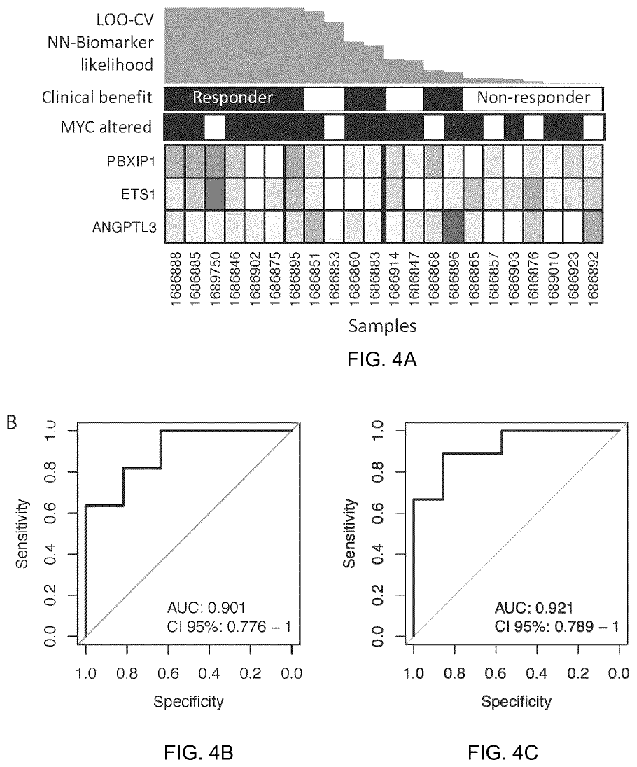

In parallel to the phase 1 and 2 clinical trials, VIPER was performed to determine if gene expression patterns correlated with activity of proteins associated with MYC as well as a biomarker pattern of clinical response. For this analysis, 22 pre-treatment tumor samples from 11 responding and 11 non-responding patients, 13 of which were MYC-altered, were included. Of note, for the 9 non-MYC-altered specimens, 5 did not undergo central testing for MYC-alterations. Significant enrichment of 67 B-cell context-specific MYC-interacting proteins was observed among the proteins most differentially active between fimepinostat responder and non-responders (p<0.001, Gene Set Enrichment Analysis [GSEA], FIG. 3). As part of a the OncoMarker biomarker discovery algorithm,²¹ a Neural-Network classifier was trained on protein activity profiles of analyzed tumor samples. The analysis identified three proteins—PBXIP1, ETS1 and ANGPTL3—as Master Regulators (MRs) of fimepinostat sensitivity (FIG. 4A), yielding optimal predictive power based on leave-one-out cross-validation (LOO-CV) (Area Under Receiver Operating Characteristic Curve [AUC]=0.901, 95% CI 0.776-1 [FIG. 4B]). The biomarker correctly identified 9 of 11 responding (82%) and misclassified only 2 of 11 non-responding (18%) patients (FIG. 4A). When restricting this analysis to 13 MYC-altered patients, the fimepinostat-sensitivity biomarker had equivalent performance (LOO-CV AUC=0.881, 95% CI 0.689-1 [FIG. 4C]) and correctly identified 5 of 6 responding (83%) and misclassified only 1 of 7 non-responding (14%) patients (FIG. 4A).

### DISCUSSION

Approximately ⅓ of patients with newly-diagnosed DLBCL/HGBL demonstrate MYC-altered disease, and these patients are at risk for treatment failure following curative-intent first- and second-line immunochemotherapy and HDC/ASCT. In a cohort of patients with R/R DLBCL/HGBL with MYC alteration as defined by central testing who were treated with dual HDAC/PI3K inhibitor fimepinostat and evaluable for response, the overall response rate was 21%. Additionally, the median duration of response in responding patients was not yet reached and 73% of responding patients were estimated to have a continued response at 6 months. Furthermore, 4 responding patients remained on treatment for over 2 years without disease progression. The median time to response was 2.8 months, with approximately 80% of patients experiencing disease progression prior to that time point, suggesting that the potential for therapeutic activity of fimepinostat may not have been realized by the majority of patients demonstrating treatment failure.

Analysis by VIPER revealed differentially active proteins in responding vs. non-responding patients were significantly enriched in B-cell context-specific MYC-interacting proteins, supporting preclinical evidence that fimepinostat treatment abrogates MYC activity via multiple mechanisms of action. Additionally, fimepinostat sensitivity was accurately predicted by the same three-protein classifier in both a cohort of tumor samples without selection by MYC alteration status, as well as a subset with MYC altered disease. The fact that the majority of these tumor samples were known to be MYC-altered, and the AUC of nearly 0.9 for this biomarker predicting clinical response in patients with MYC-altered disease provide a strong rationale to attempt to validate this finding in additional patients with MYC-altered disease treated with fimepinostat on clinical trials.

Outcomes of patients with MYC-altered DLBCL/HGBL reported in clinical trials of commercially-available therapies for R/R DLBCL are listed in Table 6.^{26,29} While the overall response rate of fimepinostat is lower than that of patients with MYC-altered disease treated with most of these therapies, the lack of reporting of survival and duration of response data for patients with MYC-altered disease raises uncertainty about the long-term benefit of these therapies for this patient population.

TABLE 6
Outcomes of MYC-altered DLBCL/HGBL patients reported in clinical
trials of commercially-available therapies for R/R DLBCL

| | | | | | Median |
|---|---|---|---|---|---|
| | Study | Patient | Sample | ORR/ | PFS/OS/ |
| Therapy | design | type | size | CRR | DOR |
| Selinexor | Prospective | MYC IHC | 47 | 13% | Not |
| | phase 2 | >40% | | (ORR) | reported |
| Tafisitamab/ | Prospective | MYC | 7 | 57%/ | Not |
| lenalidomide | phase 2 | translocation | | 43% | reported |
| Tisagenlecleucel | Prospective | DHL | 16 | 50%/ | Not |
| | phase 2 | | | 25% | reported |
| Polatuzumab/ | Prospective | DEL | 11 | 46%/ | Not |
| bendamustine/ | phase 2 | | | 36% | reported |
| rituximab | | | | | |

R/R = relapsed/refractory.
US FDA = United States Food and Drug Administration.
ORR = overall response rate.
CRR = complete response rate.
PFS = progression free survival.
OS = overall survival. DOR-duration of response.
DHL = double hit lymphoma.
IHC = immunohistochemical staining.
DEL = double expressor lymphoma.

The strengths of our analysis include central review for MYC alterations as well as robust tracking of patient outcomes and toxicities experienced through prospective data collection from clinical trial protocols. The weaknesses of our analysis include the inability to identify all patients MYC-altered disease treated on these clinical trial protocols with certainty due to lack of availability of tissue for central testing for all forms of MYC alteration in all cases, as well as a small sample size that precludes meaningful univariable and multivariable analyses which could predict for the association of baseline characteristics with disease response and/or survival.

In conclusion, objective responses were experienced following treatment with fimepinostat in R/R DLBCL/HGBL patients with MYC-altered disease, with prolonged durations of response in responding patients. These outcomes support the use of fimepinostat in this clinical setting, as well as further investigation in combination with other agents and/or in earlier lines of therapy with continued exploration of biomarkers which may predict for clinical activity, in hopes that the therapeutic effect of fimepinostat may be realized in a greater proportion of patients.

### REFERENCES

1. Meyer N, Penn L Z. Reflecting on 25 years with MYC. Nature reviews Cancer 2008; 8(12): 976-90.
2. Dang C V, O'Donnell K A, Zeller K I, Nguyen T, Osthus R C, Li F. The c-Myc target gene network. Seminars in cancer biology 2006; 16(4): 253-64.
3. Savage K J, Johnson N A, Ben-Neriah S, et al. MYC gene rearrangements are associated with a poor prognosis in diffuse large B-cell lymphoma patients treated with R-CHOP chemotherapy. Blood 2009; 114(17): 3533-7.
4. Barrans S, Crouch S, Smith A, et al. Rearrangement of MYC is associated with poor prognosis in patients with diffuse large B-cell lymphoma treated in the era of rituximab. Journal of clinical oncology: official journal of the American Society of Clinical Oncology 2010; 28(20): 3360-5.

Cuccuini W, Briere J, Mounier N, et al. MYC+diffuse large B-cell lymphoma is not salvaged by classical R-ICE or R-DHAP followed by BEAM plus autologous stem cell transplantation. Blood 2012; 119(20): 4619-24.
6. Petrich A M, Gandhi M, Jovanovic B, et al. Impact of induction regimen and stem cell transplantation on outcomes in double-hit lymphoma: a multicenter retrospective analysis. Blood 2014; 124(15): 2354-61.
7. Herrera A F, Mei M, Low L, et al. Relapsed or Refractory Double-Expressor and Double-Hit Lymphomas Have Inferior Progression-Free Survival After Autologous Stem-Cell Transplantation. Journal of clinical oncology: official journal of the American Society of Clinical Oncology 2017; 35(1): 24-31.
8. Valera A, Lopez-Guillermo A, Cardesa-Salzmann T, et al. MYC protein expression and genetic alterations have prognostic impact in patients with diffuse large B-cell lymphoma treated with immunochemotherapy. Haematologica 2013; 98(10): 1554-62.
9. Li S, Seegmiller A C, Lin P, et al. B-cell lymphomas with concurrent MYC and BCL2 abnormalities other than translocations behave similarly to MYC/BCL2 double-hit lymphomas. Modern pathology: an official journal of the United States and Canadian Academy of Pathology, Inc 2015; 28(2): 208-17.
10. Meriranta L, Pasanen A, Alkodsi A, Haukka J, Karjalainen-Lindsberg M L, Leppa S. Molecular background delineates outcome of double protein expressor diffuse large B-cell lymphoma. Blood advances 2020; 4(15): 3742-53.
11. Green T M, Young K H, Visco C, et al. Immunohistochemical double-hit score is a strong predictor of outcome in patients with diffuse large B-cell lymphoma treated with rituximab plus cyclophosphamide, doxorubicin, vincristine, and prednisone. Journal of clinical oncology: official journal of the American Society of Clinical Oncology 2012; 3460-7.
12. Johnson N A, Slack G W, Savage K J, et al. Concurrent expression of MYC and BCL2 in diffuse large B-cell lymphoma treated with rituximab plus cyclophosphamide, doxorubicin, vincristine, and prednisone. Journal of clinical oncology: official journal of the American Society of Clinical Oncology 2012; 30(28): 3452-9.
13. Hu S, Xu-Monette Z Y, Tzankov A, et al. MYC/BCL2 protein coexpression contributes to the inferior survival of activated B-cell subtype of diffuse large B-cell lymphoma and demonstrates high-risk gene expression signatures: a report from The International DLBCL Rituximab-CHOP Consortium Program. Blood 2013; 121(20): 4021-31; quiz 250.
14. Landsburg D J. Management of Patients with MYC-Altered Lymphomas. Current hematologic malignancy reports 2016; 11(3): 208-17.
15. Sun K, Atoyan R, Borek M A, et al. Dual HDAC and PI3K Inhibitor CUDC-907 Downregulates M Y C and Suppresses Growth of MYC-dependent Cancers. Molecular cancer therapeutics 2017; 16(2): 285-99.
16. Younes A, Berdej a J G, Patel M R, et al. Safety, tolerability, and preliminary activity of CUDC-907, a first-in-class, oral, dual inhibitor of HDAC and PI3K, in patients with relapsed or refractory lymphoma or multiple myeloma: an open-label, dose-escalation, phase 1 trial. The Lancet Oncology 2016; 17(5): 622-31.
17. Oki Y, Kelly K R, Flinn I, et al. CUDC-907 in relapsed/refractory diffuse large B-cell lymphoma, including patients with MYC-alterations: results from an expanded phase I trial. Haematologica 2017; 102(11): 1923-30.
18. Cheson B D, Pfistner B, Juweid M E, et al. Revised response criteria for malignant lymphoma. Journal of clinical oncology: official journal of the American Society of Clinical Oncology 2007; 25(5): 579-86.
19. Alvarez M J, Shen Y, Giorgi F, et al. Functional characterization of somatic mutations in cancer using network-based inference of protein activity. Nat Genet 2016; 48(8): 838-47.
20. Neal M. Assay Validation Review, Wadsworth Center, NY State Department of Health, PFI: 7313, Project I D: 63859. 2019.
21. Chari A, Vogl D T, Gavriatopoulou M, et al. Oral Selinexor-Dexamethasone for Triple-Class Refractory Multiple Myeloma. The New England journal of medicine 2019; 381(8): 727-38.
22. Ashburner M, Ball C A, Blake J A, et al. Gene ontology: tool for the unification of biology. The Gene Ontology Consortium. Nat Genet 2000; 25(1): 25-9.
23. Hongxu D H, Douglass E F, Sonabend A M, et al. Quantitative Assessment of Protein Activity in Orphan Tissues and Single Cells Using the metaVIPER Algorithm. Nature Methods 2018; 9(1): 1471.
24. Basso K, Margolin A A, Stolovitzky G, Klein U, Dalla-Favera R, Califano A. Reverse engineering of regulatory networks in human B cells. Nature genetics 2005; 37(4): 382-90.
25. Bishop C M. Neural Networks for Pattern Recognition: Clarendon Press, Oxford.; 1995.
26. Kalakonda N, Maerevoet M, Cavallo F, et al. Selinexor in patients with relapsed or refractory diffuse large B-cell lymphoma (SADAL): a single-arm, multinational, multicentre, open-label, phase 2 trial. The Lancet Haematology 2020; 7(7): e511-e22.
27. Salles G, Duell J, Gonzalez Barca E, et al. Tafasitamab plus lenalidomide in relapsed or refractory diffuse large B-cell lymphoma (L-MIND): a multicentre, prospective, single-arm, phase 2 study. The Lancet Oncology 2020; 21(7): 978-88.
28. Schuster S J, Bishop M R, Tam C S, et al. Tisagenlecleucel in Adult Relapsed or Refractory Diffuse Large B-Cell Lymphoma. The New England journal of medicine 2019; 380(1): 45-56.
29. Sehn L H, Herrera A F, Flowers C R, et al. Polatuzumab Vedotin in Relapsed or Refractory Diffuse Large B-Cell Lymphoma. Journal of clinical oncology: official journal of the American Society of Clinical Oncology 2020; 38(2): 155-65.
30. Hans C P, Weisenburger D D, Greiner T C, et al. Confirmation of the molecular classification of diffuse large B-cell lymphoma by immunohistochemistry using a tissue microarray. Blood 2004; 103(1): 275-82.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of classifying a subject suffering from diffuse large B cell lymphoma as a fimepinostat responder or a fimepinostat non-responder, comprising the step of determining the activity of one or more marker proteins in a tumor sample from the subject, wherein increased or decreased activity of the one or more marker proteins compared to baseline classifies the subject as a fimepinostat responder and an absence of increased or decreased activity of the one or more marker proteins compared to baseline classifies the subject as a fimepinostat non-responder.

2. The method of claim 1, wherein the activities of the one or more marker proteins are provided to a trained classifier, the trained classifier being trained to differentiate between fimepinostat responders and fimepinostat non-responders; and obtaining from the classifier the classification of the subject as a fimepinostat responder or a fimepinostat non-responder.

3. The method of claim 1, wherein the one or more marker proteins are selected from the group consisting of PBXIP1, ETS1, ANGPTL3, YEATS4, IL16, FGD3, ATF7IP, TRIP13, CBX4 and CD37.

4. The method of claim 3, wherein said method comprises the step of determining the activity of two or more marker proteins.

5. The method of claim 4, wherein said method comprises the step of determining the activity of three or more marker proteins.

6. The method of claim 1, wherein the one or more marker proteins are selected from the group consisting of PBXIP1, ETS1 and ANGPTL3.

7. The method of claim 6, wherein said method comprises the step of determining the activity of PBXIP1, ETS1 and ANGPTL3.

8. The method of claim 1, wherein the protein activity is determined using the VIPER algorithm.

9. The method of claim 1, wherein the subject is naïve to therapy with fimepinostat.

10. A method of treating diffuse large B-cell lymphoma in a subject in need thereof, comprising the step of administering to the subject a therapeutically effective amount of fimepinostat or a pharmaceutically acceptable salt thereof, wherein the subject is classified as a fimepinostat responder by the method of claim 1.

11. A method of treating diffuse large B-cell lymphoma in a subject in need thereof, comprising:
(a) classifying the subject as a fimepinostat responder or a fimepinostat non-responder by the method of claim 1; and
(b) (i) if the subject is classified as a fimepinostat responder, administering to the subject a therapeutically effective amount of fimepinostat or a pharmaceutically acceptable salt thereof; and
(ii) if the subject is classified as a fimepinostat non-responder, administering to the subject a therapeutically effective amount of a therapy for diffuse large B-cell lymphoma which is not fimepinostat or a pharmaceutically acceptable salt thereof.

12. A method of treating DLBCL in a subject in need thereof, wherein the subject is a fimepinostat responder, comprising the steps of (a) receiving information identifying the subject as a fimepinostat responder; and (b) administering to the subject a therapeutically effective amount of fimepinostat or a pharmaceutically acceptable salt thereof.

13. The method of claim 10, wherein the fimepinostat is administered as the methanesulfonate salt or the benzenesulfonate salt.

14. The method of claim 12, wherein the fimepinostat or pharmaceutically acceptable salt thereof is orally administered.

15. The method of claim 14, wherein the fimepinostat or pharmaceutically acceptable salt thereof is administered at a daily dose of 60 mg free base equivalent.

16. The method of claim 14, wherein the fimepinostat or pharmaceutically salt thereof is administered at a dose of 60 mg free base equivalent for five days, followed by no fimepinostat or pharmaceutically acceptable salt thereof for two days.

17. The method of claim 15, wherein the fimepinostat is administered as the methanesulfonate salt.
